(19) United States
(12) Patent Application Publication
(10) Pub. No.: US 2019/0262563 A1
(43) Pub. Date: Aug. 29, 2019

(54) ARTIFICIAL AIRWAY DEVICE

(71) Applicant: TELEFLEX LIFE SCIENCES UNLIMITED COMPANY, Hamilton (BM)

(72) Inventor: Kien Chung KWOK, Singapore (SG)

(21) Appl. No.: 16/342,891

(22) PCT Filed: Oct. 20, 2017
(86) PCT No.: PCT/EP2017/076922

§ 371 Date: Apr. 17, 2019

(30) Foreign Application Priority Data
Oct. 21, 2016 (GB) 1617855.0

(51) Int. Cl. A61M 16/04

(52) U.S. Cl. CPC A61M 16/0415; A61M 16/0409; A61M 16/047; A61M 16/0445; A61M 16/0463

(57) ABSTRACT

An artificial airway device 1 to facilitate lung ventilation of a patient, comprising an airway tube 2 including an airway lumen 3, a mask 4 at one end of the airway tube, the mask including a backplate 5 and having a peripheral formation 6 capable of forming a seal around the circumference of the laryngeal inlet, the peripheral formation surrounding a hollow interior space or lumen 7 of the mask and the airway lumen 3 opening into the lumen of the mask, wherein the airway tube 2 comprises an outer tube part 201 and an inner core 202, the inner core 202 defining partly or completely the airway lumen.

The present invention relates to an improved artificial airway device, and in particular to an improved laryngeal mask and method for manufacturing same.

For at least seventy years, endotracheal tubes comprising a long slender tube with an inflatable balloon disposed near the tube's distal end have been used for establishing airways in unconscious patients. In operation, the endotracheal tube's distal end is inserted through the mouth of the patient, into the patient's trachea. Once positioned, the balloon is inflated so as to form a seal with the interior lining of the trachea. After this seal is established, positive pressure may be applied to the tube's proximal end to ventilate the patient's lungs. Also, the seal between the balloon and the inner lining of the trachea protects the lungs from aspiration (e.g., the seal prevents material regurgitated from the stomach from being aspirated into the patient's lungs).

Although they have been successful, endotracheal tubes suffer from several major disadvantages. The principal disadvantage of the endotracheal tube relates to the difficulty of properly inserting the tube. Inserting an endotracheal tube into a patient is a procedure that requires a high degree of skill. Also, even for skilled practitioners, insertion of an endotracheal tube is sometimes difficult or not possible. In many instances, the difficulty of inserting endotracheal tubes has tragically led to the death of a patient because it was not possible to establish an airway in the patient with sufficient rapidity. Also, inserting an endotracheal tube normally requires manipulation of the patient's head and neck and further requires the patient's jaw to be forcibly opened widely. These necessary manipulations make it difficult, or undesirable, to insert an endotracheal tube into a patient who may be suffering from a neck injury.

The laryngeal mask airway device is a well known device that is useful for establishing airways in unconscious patients, and which seeks to address some of the known drawbacks associated with endotracheal tubes.

In contrast to the endotracheal tube, it is relatively easy to insert a laryngeal mask airway device into a patient and thereby establish an airway. Also, the laryngeal mask airway device is a “forgiving” device in that even if it is inserted improperly, it still tends to establish an airway. Accordingly, the laryngeal mask airway device is often thought of as a “life saving” device. Also, the laryngeal mask airway device may be inserted with only relatively minor manipulation of the patient's head, neck and jaw. Further, the laryngeal mask airway device provides ventilation of the patient's lungs without requiring contact with the sensitive inner lining of the trachea and the internal diameter of the airway tube is typically significantly larger than that of the endotracheal tube. Also, the laryngeal mask airway device does not interfere with coughing to the same extent as endotracheal tubes. Largely due to these advantages, the laryngeal mask airway device has enjoyed increasing popularity in recent years.

U.S. Pat. No. 4,509,514 describes a laryngeal mask airway device which consists of the basic parts which make up most if not all laryngeal mask airway devices, namely an airway tube opening at one end into the interior of a hollow mask portion shaped to fit readily behind the larynx of a patient. The periphery of the mask is formed by a cuff which in use forms a seal around the opening of the larynx. This enables the airway to be established effectively.

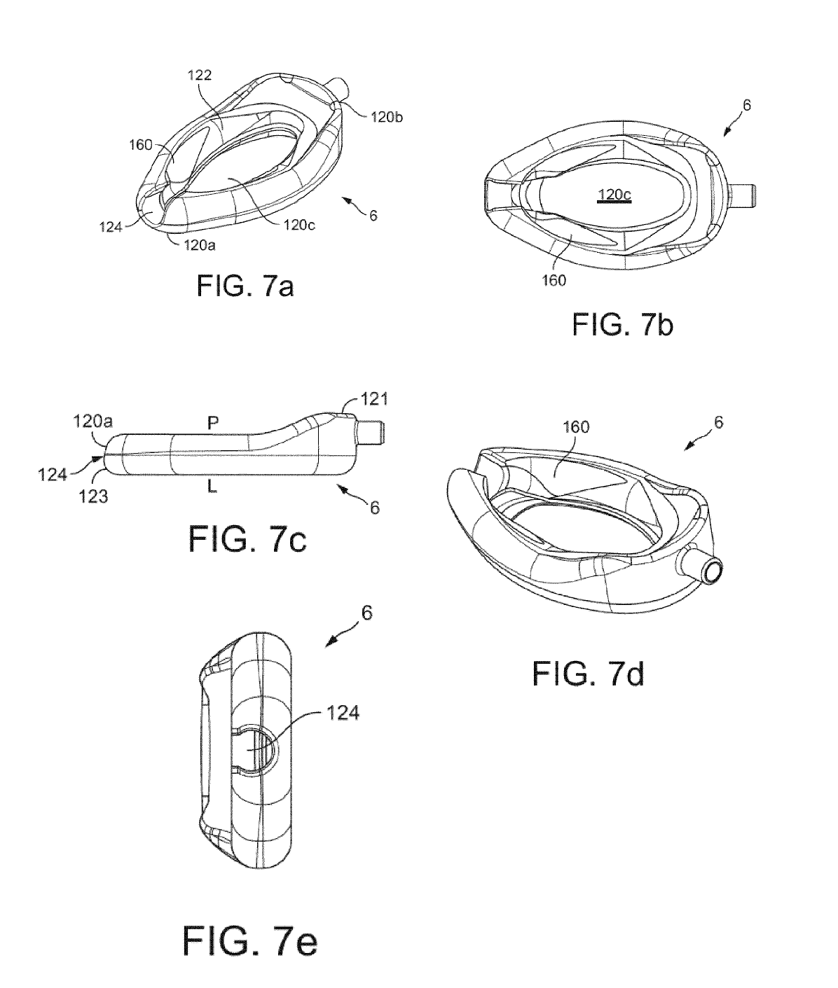

Laryngeal mask airway devices with specific provision for gastric-discharge drainage have been developed, as exemplified by U.S. Pat. No. 4,995,388 (FIGS. 7 to 10); U.S. Pat. Nos. 5,241,956; and 5,355,879. These devices generally incorporate a small-diameter drainage tube having an end located at the distal end of the mask, so as to lie against the upper end of the upper oesophageal sphincter when the mask is in place, the tube being of sufficient length to extend out of the mouth of the patient to enable active or passive removal of gastric discharge from the upper oesophageal sphincter. According to alternative proposals, the drainage tube may extend beyond the distal end of the mask, into the oesophagus itself (U.S. Pat. No. 4,995,388, FIGS. 7 and 11).

Laryngeal mask airway devices are now commonly used to aid in insertion of endotracheal tubes, and such devices are referred to as intubating laryngeal masks, an example being Applicant's own “Fastrach”™ device.

Since the original invention of the laryngeal mask airway device by Dr Archibald Brain, numerous techniques for manufacturing them have been used. In the early days devices were made by assembling pre-formed components, mainly because different materials were used for the different components, but these processes were labour intensive and time consuming and quality control was difficult. A particularly difficult aspect was the formation of the inflatable cuff, especially after the invention by Dr Brain of so called “second generation” devices that include the provision of a second lumen in addition to the airway lumen for drainage of gastric matter. In order to be most effective it was found that the inlet to the gastric drain should be positioned adjacent the distal end of the mask, which meant that the drain would need to pass through the cuff, potentially compromising its integrity. With the advent of PVC devices one of the most commercially successful and widely adopted techniques was injection molding. Components of the device are molded separately and attached together by separate manufacturing steps or in some cases, devices are molded in one piece, usually with a final finishing step, and these techniques are the most widely used today. A problem experienced by manufacturers concerns the fact that since its inception the original, basic laryngeal mask design has diversified into many specialist forms with varying characteristics. For example, there are simple, “Classic” type devices, paediatric orientated devices, intubating devices, and “second generation” devices, to name a few. This makes manufacture more difficult and more and more expensive. In the case of integrally molded designs, separate tools are required for each design. In the case of devices made from separately molded parts, the number of different parts now required to be made and kept for assembly is high. In both cases, for relatively specialist, low sales volume devices the costs of manufacture can make the devices prohibitively expensive, particularly for poorer countries. Efforts to overcome these challenges have centred mainly on techniques for simplifying cuff formation.

The present invention seeks to ameliorate problems associated with the prior-art described above.

According to a first aspect of the invention, there is provided an artificial airway device to facilitate lung ventilation of a patient, comprising an airway tube including an airway lumen, a mask at one end of the airway tube, the mask including a backplate and having a peripheral formation capable of forming a seal around the circumference of the laryngeal inlet, the peripheral formation surrounding a hollow interior space or lumen of the mask and the airway lumen opening into the lumen of the mask, wherein the airway tube comprises an outer tube part and an inner core, the inner core defining partly or completely the airway lumen.

The inner core may further define partly or completely one or more additional lumen. Said one or more additional lumen may be adapted to receive a sensor or viewing device. In particular, said additional lumen may include a recess for location of a sensor. Said one or more additional lumen may further include one or more lumen disposed to allow in use, for access to the oesophageal sphincter of the patient and/or removal of gastric fluid. The or each additional lumen may be defined entirely by the inner core, or by a combination of the inner core and the outer tube part.

Typically, at least one groove is provided on the outer surface of the inner core. In one embodiment, two grooves may be provided on the outer surface of the inner core, typically on opposite sides of the outer surface of the inner core Preferably, the at least one groove provided on the outer surface of the inner core and an inner wall of the outer tube part form at least one lumen when the inner core is inserted into the outer tube part.

Preferably, the outer tube takes the form of a tube having a fixed curve portion. It is preferred that the outer tube further comprises a straight portion and a backplate portion. Preferably, the outer tube takes the form of a tube having a straight portion, a fixed curve portion and a backplate portion moving from the proximal to distal end. Preferably, the outer tube comprises a through bore running throughout from its proximal to distal ends.

Preferably, the inner surface of the outer tube comprises raised guide tracks. Preferably, the raised guide tracks extend from near the proximal end to the distal end of the straight portion.

It is preferred that the outer surface of the inner core comprises at least one further groove. Preferably, the at least one further groove provided on the outer surface of the inner core may engage with a corresponding track provided on the inner surface of the outer tube. Advantageously, engagement of the at least one track and at least one further groove assists in securing the inner core within the outer tube part.

In another embodiment, at least one track may be provided on the outer surface of the inner core and/or at least one further groove may be provided on the inner surface of the outer tube, wherein the at least one track may engage with a corresponding further groove. Advantageously, engagement of the at least one track and at least one further groove assists in securing the inner core within the outer tube part.

Preferably, the inner core is dimensioned to fit inside the outer tube part. Typically, the inner core extends substantially along the entire length of the outer tube part. It is preferred that the inner core comprises an inner backplate portion.

Preferably, the airway tube comprises silicone or PVC. Preferably, the outer tube part and/or the inner core comprise silicone or PVC.

In one embodiment, the sensor may be a temperature sensor. Preferably, the temperature sensor comprises a thermistor. Typically, the temperature sensor may be provided on the airway tube. In one embodiment, the temperature sensor may be provided on the inner core. In another embodiment, the temperature sensor may be provided on the outer tube part. In one embodiment, the temperature sensor may comprise a sensor tip, a lead wire and a connector, wherein the connector may be a moulded connector. Typically, temperature display and logging are achieved by plugging the connector part of the temperature sensor into a patient monitor. In one embodiment, the sensor tip is encased within the wall of the airway tube along the anterior surface of the tube. Typically, the sensor tip is encased within the wall of the airway tube along the anterior surface of the tube that rests against the pharyngeal portion of the tongue when the device is inserted within a patient. Preferably, the temperature sensor measures the temperature within the oropharynx of the patient. In one embodiment, the lead wire of the temperature sensor runs along the airway tube, extends out of the airway connector and terminates at the sensor connector. In one embodiment, the lead wire of the temperature sensor runs along the inner surface of the airway tube. Advantageously, the temperature sensor may be used to measure the core temperature of a patient.

It is preferred that the peripheral formation comprises an inflatable cuff, or a non-inflatable cuff. It is further preferred that where the peripheral formation comprises an inflatable cuff, the backplate overlies the cuff and is bonded to it, such that on deflation the cuff may be collapsed upon it, thereby encouraging the cuff to pack flat.

According to a second aspect of the invention, there is provided a method of treating a patient using a device as defined hereinabove.

According to a third aspect of the invention, there is provided a method of manufacturing an airway tube for a laryngeal mask airway device, the method comprising the steps of forming an airway tube by providing an inner core and an outer tube part, the inner core defining an airway lumen, and inserting the inner core into the outer tube part.

It is preferred that the method includes the step of forming the inner core by injection moulding. As an alternative, the method may include the step of forming the inner core by extrusion.

It is preferred that the method includes the step of forming the outer tube by injection moulding. As an alternative, the method may include the step of forming the outer tube by extrusion.

The invention will now further be described by way of example, with reference to the accompanying drawings, in which:

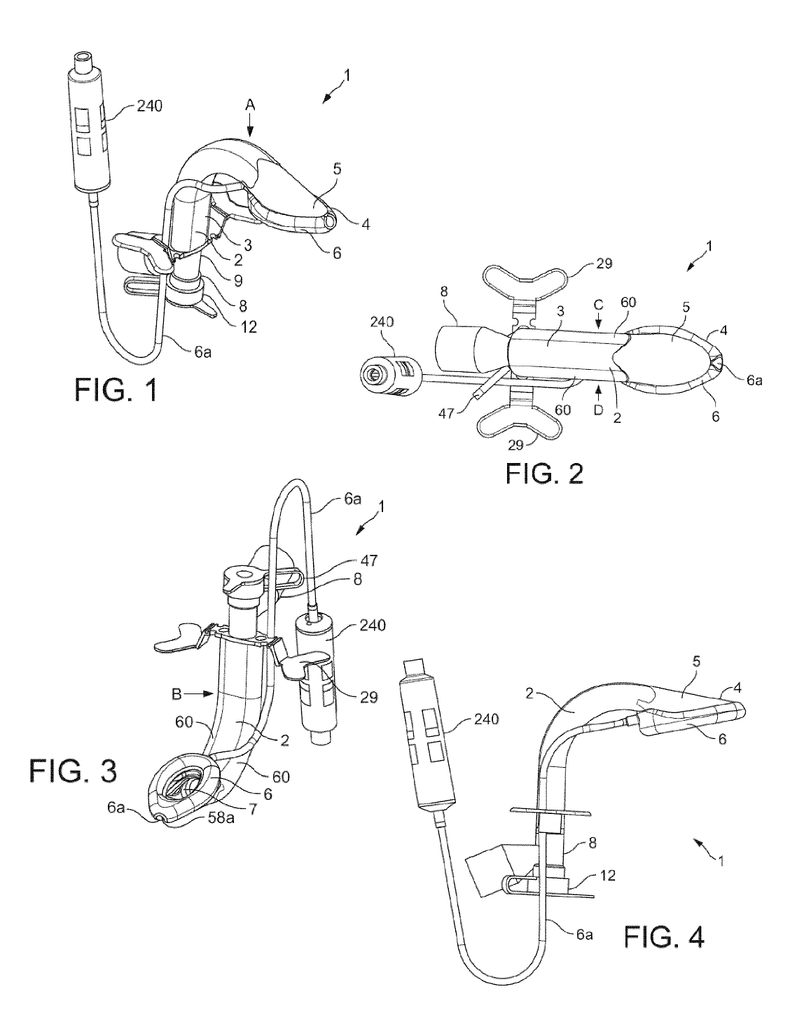

FIG. 1 is a dorsal isometric view of a device according to the invention;

FIG. 2 is a dorsal view of the device of FIG. 1;

FIG. 3 is a ventral isometric view of the device of FIG. 1;

FIG. 4 is a left side view of the device of FIG. 1;

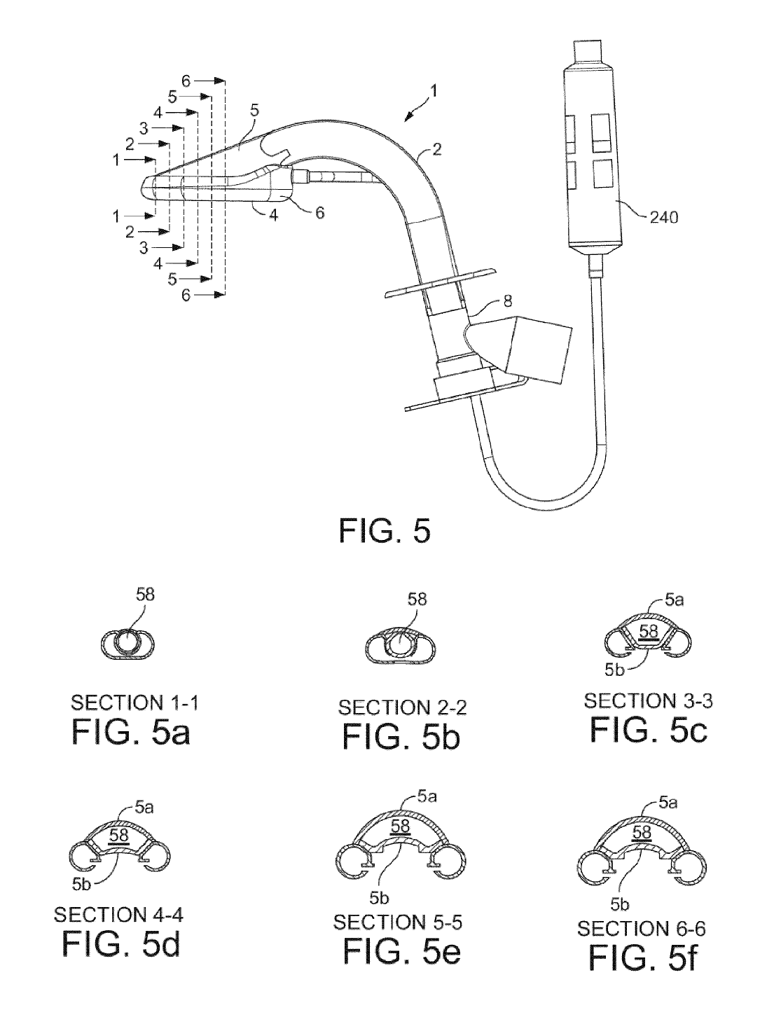

FIG. 5 is a right side view of the device of FIG. 1;

FIGS. 5a to 5f are transverse sectional views along long lines 1-1 to 6-6 in FIG. 5;

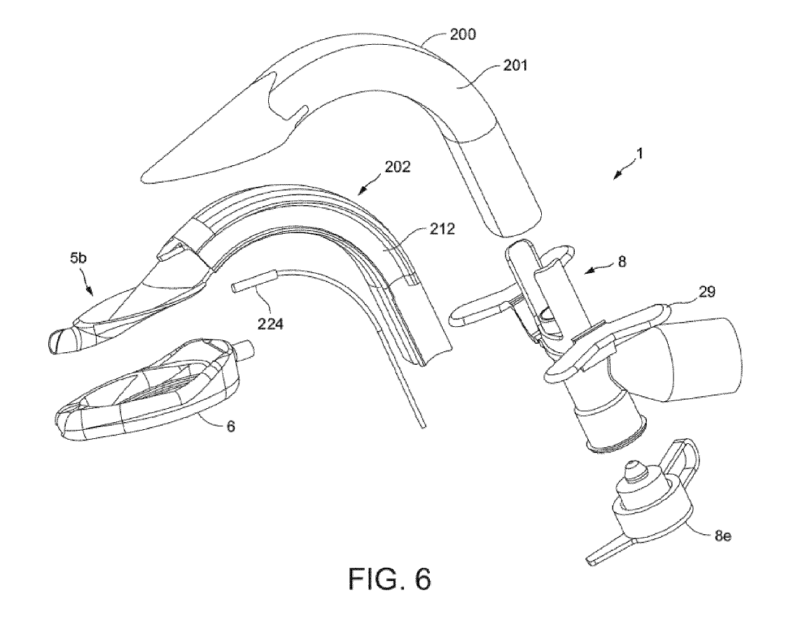

FIG. 6 is a right side exploded view of a device according to the invention;

FIG. 7a is a front isometric view of a part of the device of FIG. 6;

FIG. 7b is a dorsal view of the part shown in FIG. 7a;

FIG. 7c is a right side view of the part shown in FIG. 7a;

FIG. 7d is a rear isometric view of the part shown in FIG. 7a;

FIG. 7e is a front view of the part shown in FIG. 7a;

FIG. 8 is a dorsal view of a part of the device shown in FIG. 6;

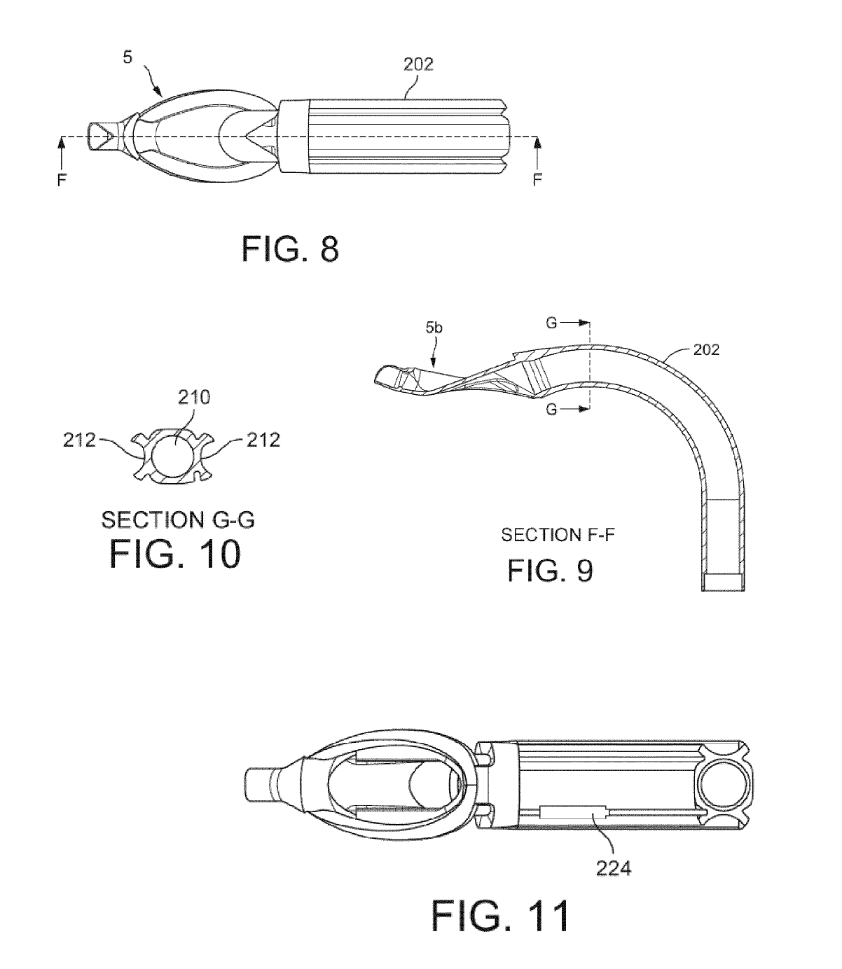

FIG. 9 is a longitudinal sectional view along line F-F in FIG. 8;

FIG. 10 is a transverse sectional view along line G-G in FIG. 9;

FIG. 11 is a ventral view of the part shown in FIG. 8;

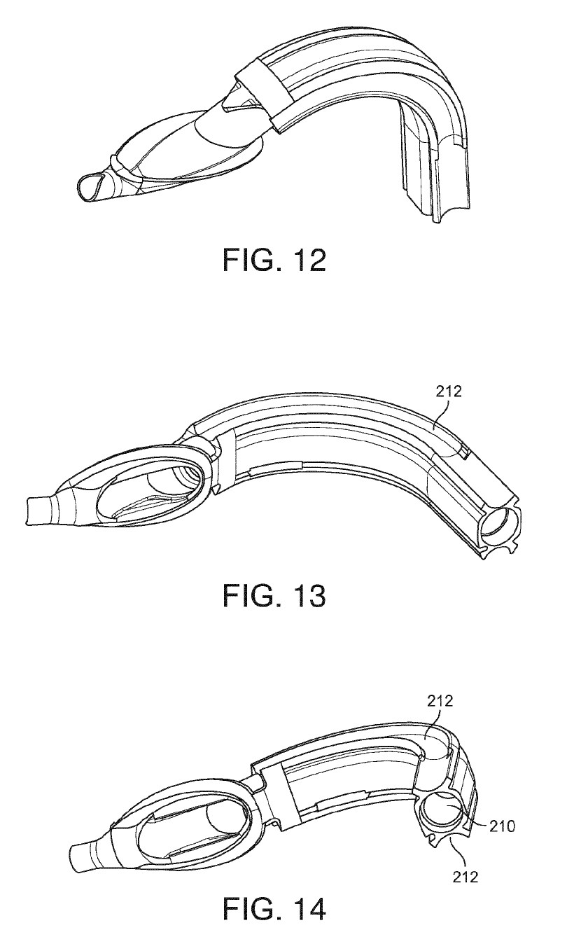

FIG. 12 is a front dorsal isometric view of the part shown in FIG. 8;

FIG. 13 is a right side ventral isometric view of the part shown in FIG. 8;

FIG. 14 is a ventral view of the part shown in FIG. 8;

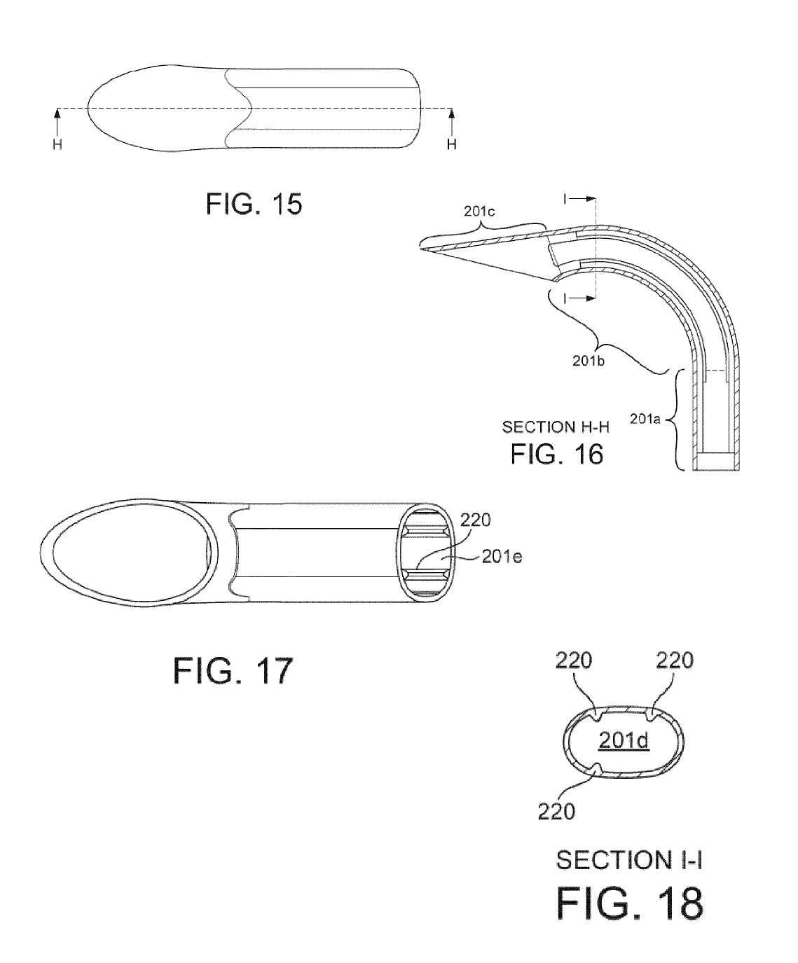

FIG. 15 is a dorsal view of a further part of the device shown in FIG. 6;

FIG. 16 is a longitudinal sectional view along line H-H in FIG. 15;

FIG. 17 is a ventral view of the part shown in FIG. 15;

FIG. 18 is a transverse sectional view along line I-I in FIG. 16;

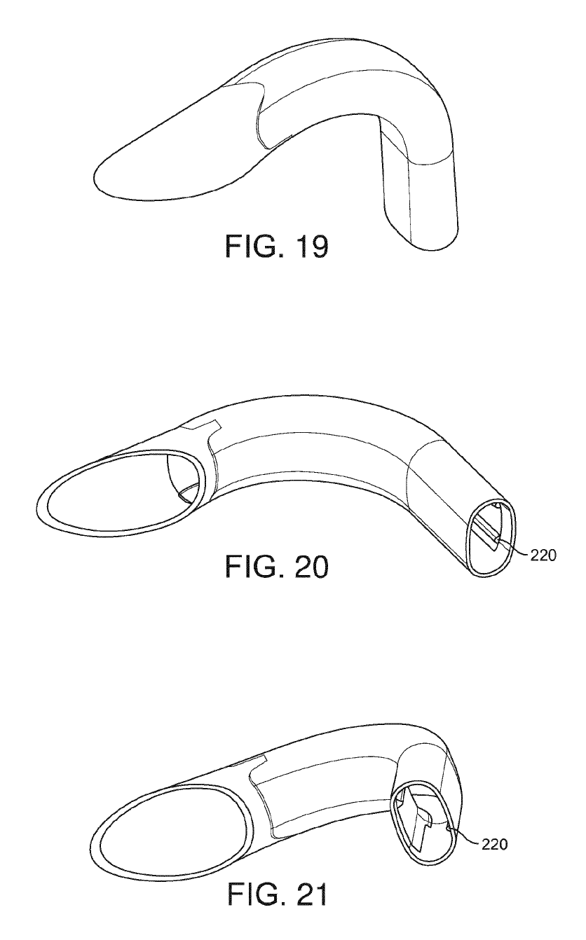

FIG. 19 is a front dorsal isometric view of the part shown in FIG. 15;

FIG. 20 is a right side ventral isometric view of the part shown in FIG. 15;

FIG. 21 is a right side rear ventral isometric view of the part shown in FIG. 15;

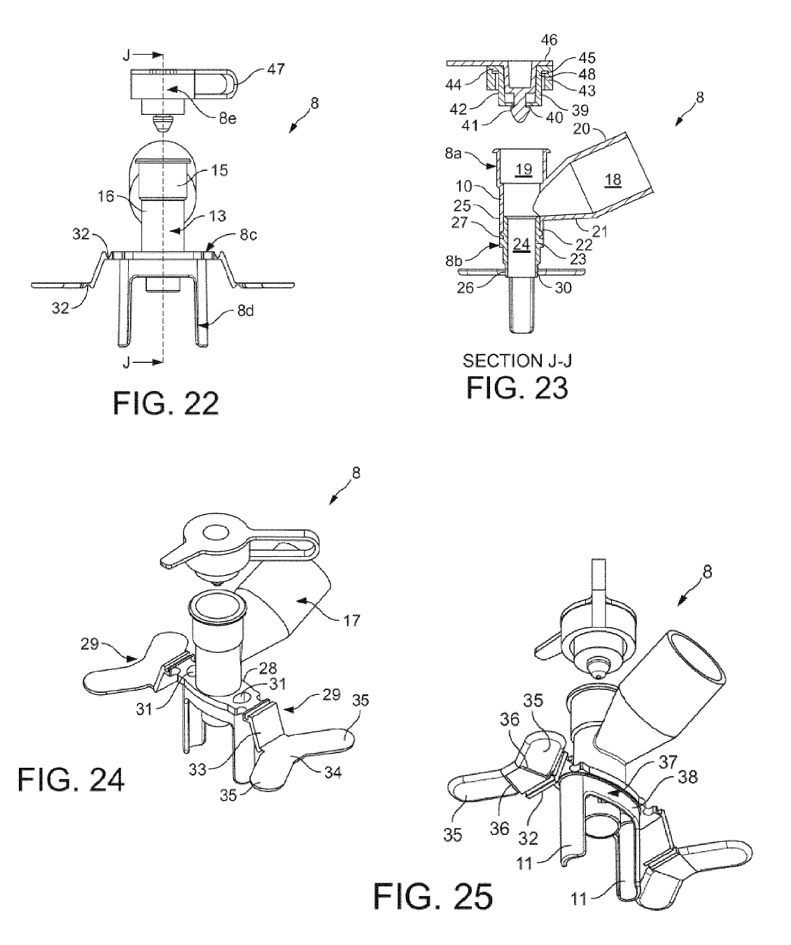

FIG. 22 is a front view of the connector shown in FIG. 6;

FIG. 23 is a longitudinal sectional view along line J-J in FIG. 22;

FIG. 24 is a top plan isometric view of the connector shown in FIG. 6; and

FIG. 25 is an under plan isometric view of the connector shown in FIG. 6.

In the discussion of the following exemplary embodiments, like parts will generally be given the same reference numerals throughout the description.

For convenience of exposition, referring to FIGS. 1 to 4, reference letter A denotes the dorsal surface of the device. Reference letter B denotes the ventral surface of the device. In accordance with standard practice, that part of the device 1 that in use will extend from the patient is referred to herein as the proximal end (in the sense that it is nearest the user) with the other end being referred to as the distal end. In FIG. 2, reference letter C denotes the right side and reference letter D denotes the left side.

Referring to FIGS. 1 to 6, there is illustrated an artificial airway device 1 to facilitate lung ventilation of a patient, comprising an airway tube 2 including an airway tube lumen 3, a mask 4 at one end of the airway tube, the mask including a backplate 5 and having a peripheral formation 6 capable of forming a seal around the circumference of the laryngeal inlet, the peripheral formation surrounding a hollow interior space or lumen 7 of the mask and the airway lumen 3 opening into the lumen of the mask 4, wherein the airway tube 2 comprises an outer tube 201 and an inner core 202, the inner core 202 defining partly or completely the airway lumen.

The device 1 as illustrated further comprises a connector 8 disposed at the proximal end of the airway tube, the connector 8 including a main bore 9 for passage of gas to the airway tube lumen 3, the main bore including a wall defining a circumference and including a plurality of ports 12 to allow passage into the main bore, at least one port 12 being disposed for circumferential rotational movement about the main bore 9.

At its distal end, airway tube 2 is attached to mask 4. It will be noted that airway tube 2 terminates towards the proximal end of mask 4. Thus mask 4 does not suffer in terms of being made too rigid by the material of the airway tube.

With reference to FIG. 6, an exploded view is shown, from which it can be seen that device 1 includes an airway tube 2 that comprises what is in effect, an airway tube and backplate combination part 200. The airway tube and backplate combination part 200 comprises two pieces: an outer tube 201 and an inner core 202.

Outer tube 201 is illustrated in detail in FIGS. 15 to 21. From these it can be seen that the outer tube takes the form of a tube having a straight portion 201a, a fixed curve portion 201b and a backplate portion 201c moving from the proximal to distal end. In transverse section the tube is compressed rather than circular (FIG. 18) as is know in the art, with a through bore 201d running throughout from its proximal to distal ends. As illustrated, for example, in FIGS. 16, 17 and 18, the inner surface 201e of the sheath 201 comprises three raised guide tracks 220 which extend from near the proximal end to the distal end of the straight portion 201a, one on the ventral inner surface and two on the opposing dorsal inner surface.

As mentioned, at its distal end outer tube 201 includes backplate portion 201c. One notable feature of the present invention is the construction of the backplate 5. As the skilled worker will appreciate, the term “backplate”, when used in the present technical field has come to denote that part of the mask that is surrounded by the cuff in the assembled device and which provides separation between the laryngeal and pharyngeal regions when the device is in situ in the patient. Supply of gas takes place through an aperture in the backplate via a fluid tight connection between the part of the backplate defining the aperture and the airway tube. In one known arrangement the backplate and airway tube are formed integrally which is a particularly convenient arrangement. In the prior art, backplates are generally bowl or dome shaped structures rather than flat structures and the term is therefore not entirely descriptive of the shape. In the presently described device 1, the outer tube 201 provides a part of the backplate, in particular, backplate portion 201c that acts an outer cover or skin, as illustrated in FIG. 6. Thus, backplate 5 comprises inner and outer skins 5a, 5b that together define a space there between, as shown schematically in FIGS. 5a to 5f. The space so defined is atrium 58 from which proximally, drain tubes 60 lead off and distally, inlet 58a enters. The atrium can be regarded as a manifold that connects the single gastric inlet 58a with the gastric drain tubes 60.

As mentioned above, mask 4 includes peripheral formation 6 which in this embodiment takes the form of an inflatable cuff of generally known form. Cuff 6 includes an inflation line 6a at its proximal end and has a gastric inlet aperture 6b at its distal end (FIG. 3). Referring to the exploded view in FIG. 5, it can be seen that the dorsal surface of cuff 6 is bonded to backplate 5 so that the material of the dorsal surface of the cuff 6 forms a bridge between the inner and outer skins 5a, 5b thus closing off the ventral side of atrium 58 except where gastric inlet aperture 6b enters the cuff. Thus it can be seen that gastric inlet 6b is in fluid communication with atrium 58. In an alternative method of construction the cuff 6 may be formed with a web across its aperture that itself forms the ventral surface of atrium 58.

Referring now to FIGS. 6 and 8 to 14, there is illustrated inner core 202. The inner core 202 is dimensioned to fit inside the outer tube part 201 and typically extends substantially along the entire length of the outer tube part 201. The inner core 202 also comprises an inner backplate portion. The inner core 202 comprises a tube and defines partly or completely the airway lumen 210 (see FIG. 10). The inner core 202 further defines partly or completely one or more additional lumen or groove 212. The one or more additional lumen 212 may be adapted to receive a sensor or viewing device, for example, the additional lumen may include a recess for location of a sensor. The one or more additional lumen may further include one or more lumen to allow in use, for access to the oesophageal sphincter of the patient and/or removal of gastric fluid. The one or more additional lumen may be defined entirely by the inner core 202, or by a combination of the inner core 202 and the outer tube part 201. Thus, the inner core element 202 allows a plurality of conduits to be defined within the airway tube and backplate combination part, allowing for passage of gastric matter, introduction of sensors or viewing devices, etc. The device may also be used with an endotracheal tube.

Insertion of the inner core 202 within the outer tube 201 provides an airway tube and backplate combination 200 comprising an airway lumen 210 provided at the centre of the inner core 202 and at least one additional lumen 212 which may be provided as a gastric conduit.

In the embodiment shown, for example in FIGS. 6 and 10, the inner core comprises two lumens, the lumens extending along the left and right sides of the inner core element 202. The lumens are provided in the form of a groove provided on the outer surface of the inner core 202. In this embodiment, when the inner core 202 is inserted within the outer tube 201, the combination of the lumens 212 of the inner core element 202 and the inner walls of the outer tube 201 form gastric conduits for passage for passage of gastric matter.

The inner core 202 may comprise at least one further groove or recess on the outer surface thereof, wherein the at least one further groove may engage with the at least one track 220 provided on the inner surface of the outer tube. The provision of at least one track 220 on the inner surface of the outer tube and/or a corresponding further groove on the outer surface of the inner core guides and facilitates insertion of the inner core element 202 and may further provide a means for securing the inner core 202 in place within the outer tube 201.

As shown, for example, in FIG. 11, the inner core 202 may define an additional lumen adapted to receive a sensor or viewing device (224), as shown for example in FIG. 11. In one embodiment, the sensor may be a temperature sensor. Preferably, the temperature sensor comprises a thermistor. Typically, the temperature sensor may be positioned on the airway tube. In one embodiment, the temperature sensor may be positioned on the inner core part of the airway tube. In another embodiment, the temperature sensor may be positioned on the outer tube part of the airway tube. Typically, the temperature sensor may comprise a sensor tip, a lead wire and a connector, wherein the connector may be a moulded connector. Temperature display and logging are typically achieved by plugging the connector part of the temperature sensor into a patient monitor. In one embodiment, the sensor tip is encased within the wall of the airway tube along the anterior surface. Typically, the sensor tip is encased within the wall of the airway tube along the anterior surface that rests against the pharyngeal portion of the tongue when the device is inserted within a patient. Preferably, the temperature sensor measures the temperature within the oropharynx of the patient. In one embodiment, the lead wire of the temperature sensor runs along the inner surface of the airway tube, extends out of the airway connector and terminates at the sensor connector. Advantageously, the temperature sensor may be used to measure the core temperature of a patient.

The airway tube may be formed by fitting together the inner core 202 and the outer tube part 201, wherein the inner core is inserted into the outer tube part 202. When the inner core element 202 is inserted within the outer tube part 201, the inner core element provides strength and rigidity to the airway tube and backplate combination part.

Airway tube 2 and its components is formed from a material such that it is not collapsible and has a preformed fixed curve as illustrated in FIG. 1. As an example, the airway tube 2 may be of 80 Shore A durometer according to ASTM 2240. The airway tube may be formed from any known suitable material such as PVC or silicone.

Referring now to FIGS. 7a to 7e, there is illustrated peripheral formation 6 which in this embodiment takes the form of an inflatable cuff. It will be noted that unlike many other laryngeal mask airway devices the cuff 6 is formed integrally as a separate part from the rest of the device, making it easier both to manufacture and attach to the device 1. The cuff 6 comprises a generally elliptical body with a narrower distal end 120a, a wider proximal end 120b and a central elliptical through-aperture 120c. As such it will be appreciated that the cuff resembles a ring. As can be seen from the sectional view in FIG. 7c, the elliptical body comprises a wall 123 that is generally circular in section at the distal end but deeper and irregularly shaped at the proximal end by virtue of an integrally formed extension 121 formed on the dorsal surface at the proximal end 120b. This dorsal surface extension 121 defines the proximal portion of an attachment surface 122 (FIGS. 6 and 7a). The attachment surface 122 extends from the proximal end to the distal end around the entire dorsal inner circumference of the ring. At its distal end 120a the cuff has a cylindrical through bore 121 the axis of which extends in line with the midline of the ellipse and is angled upwardly as viewed in FIG. 7c relative to the plane of the body, in other words from the ventral towards the dorsal side or when the device 1 is in use from the laryngeal to the pharyngeal side of the anatomy (L and P in FIG. 7c). The result is a circular section aperture through the cuff wall 123. The proximal end 120b of the cuff includes a port 124 that lets into the interior of the bore and the cuff. As illustrated, for example, in FIGS. 7a, 7b and 7d, the cuff comprises side projections 160 which help to prevent the occlusion of the airway by supporting the anatomy of the patient.

Connector 8 is illustrated in detail in FIGS. 22 to 25. Referring to FIGS. 22 and 23, connector 8 comprises five parts, namely access port part 8a, main bore part 8b, fixation part 8c, insert part 8d and plug 8e. With the exception of the plug 8e, each part may be injection moulded from polypropylene or polyethylene. Plug 8e is preferably formed from silicone by liquid injection moulding, transfer moulding or compression moulding.

Access port part 8a comprises a main tube 13 including a generally cylindrical wall 10 having a bore 19 and respectively an outer larger diameter part 15, an inner smaller diameter part 16, and a branch tube 17. Branch tube 17 defines branch bore 18 and is attached to inner smaller diameter part 16 such that branch bore 18 is in fluid communication with bore 19. Branch tube 17 includes an outer constant diameter section 20 that is dimensioned to connect to a standard gas supply. Constant diameter section 20 is connected to a frustoconical section 21 that in turn connects to wall 10. Inner smaller diameter part 16 includes inner circumferential groove 22 adjacent distal end.

Main bore part 8b comprises a tubular wall 23 defining a bore 24 and proximal and distal ends 25, 26. Proximal end 25 is dimensioned to be received within bore 19 of access port part 8a and includes outer circumferential ridge 27 that is dimensioned to fit into inner circumferential groove 22 of access port part 8a.

Fixation part 8c comprises generally rectangular plate 28, and fixation tabs 29. Plate 28 includes a central through-bore 30 and two side through-bores 31 which extend between the major surfaces of the plate. Fixation tabs 29 extend from the minor end surfaces of the plate 28, and are hingedly attached thereto by webs 32. Each fixation tab 29 comprises a connector plate 33, a lower plate 34 and tabs 35. As viewed in FIGS. 32 to 35 and when in use in a patient, connector plate 33 depends downwardly from its proximal hinged attachment point at a minor end surface of plate 28 at a resting angle of greater than 90 degrees thereto. At its distal end each connector plate is further hingedly attached to a lower plate 34, the surface of which is disposed at rest substantially parallel to, but at a lower level than, the surface of plate 28. Each lower plate 34 comprises two tabs 35 which are co-planar with plate 34 at rest and hingedly attached thereto via hinge points 36 (FIG. 25).

Referring to FIG. 25, insert part 8d comprises an ellipsoidal mounting ring 37 having a circumferential wall 38 and depending legs 11. Each depending leg 11 comprises an arcuate wall.

Referring to FIG. 23, plug 8e comprises a circular cup insert 39 that is dimensioned to fit via an interference fit into bore 19 of access port 8a. Insert 39 includes a bottom surface 40 with a centrally disposed through-bore 41 and a circumferential wall 42. Wall 42 includes a circumferential skirt 43 depending from its upper, as viewed edge 44, thereby defining a downwardly open channel 45 between skirt and wall. Plug 8e further comprises cap 46 which is attached by retaining strap 47 to skirt 43 and is dimensioned to fit within cup insert 39. Cap 46 includes depending knob 48 which fits within through-bore 41 when the cap is in place in the plug.

The parts are assembled by forming a connector 8 comprising an access port part 8a, main bore part 8b, fixation part 8c and insert part 8d. The plug component 8e of the connector comprises a circular cup insert 39 that is dimensioned to fit via an interface fit into bore 19 of access port 8a. The plug 8e is attached by a retaining strap 47 to skirt 43 and is dimensioned such that it fits within cup insert 39. Cap 46 including a depending knob 48 fits within through bore 41 when the cap is in place in the plug.

The connector 8 is inserted into the airway tube by inserting the insert part 8d into a recess provided at the distal end of the airway tube 2. The insert part 8d comprises depending legs 11, each depending leg 11 comprising an arcuate wall and being dimensioned such that the insert part 8d fits within the recess of the airway tube. The insert part of the connector passes through the central through-bore 30 of the fixation part 8c. The fixation part 8c is positioned at the distal end of the airway tube, wherein the major surface of the plate 28 extends along a length which is substantially perpendicular to the longitudinal axis of the laryngeal mask airway device. Fixation tabs 29 extend from the minor surfaces of the plate 28 and are hingedly attached thereto by webs 32. Each fixation tab comprises a connector plate 33, a lower plate 34 and tabs 35. With reference to FIGS. 22 to 25 and when in use in a patient, the connector plate 33 depends downwardly from its proximal hinged attachment point at a minor end surface of plate 28 at a resting angle of greater than 90 degrees thereto. At its distal end, each connector plate is further hingedly attached to a lower plate 34, the surface of which is disposed at rest substantially parallel to, but at a lower level than, the surface of plate 28. Each lower plate comprises two tabs 35 which are co-planar with plate 34 at rest and hingedly attached thereto via hinge points (FIG. 25).

With reference to FIGS. 1 to 6, there is provided a method of manufacturing an airway tube 2 for a laryngeal mask airway device 1, the method comprising the steps of forming an airway tube 2 by providing an inner core 202 and an outer tube part 201, the inner core 202 defining an airway lumen 210, and inserting the inner core into the outer tube part.

The method may include the step of forming the inner core by injection moulding. As an alternative, the method may include the step of forming the inner core by extrusion.

The method may include the step of forming the outer tube by injection moulding. As an alternative, the method may include the step of forming the outer tube by extrusion.

In use, the device 1 is inserted into a patient to establish an airway as with prior art devices. Insertion is effected to the point where gastric inlet aperture 6b meets the patient's oesophageal sphincter, thus establishing fluid communication therebetween. If vomiting or regurgitation occurs, as with previous gastric access laryngeal masks, the material from the oesophagus passes into gastric inlet aperture 6b. However, unlike with previous devices the material passes into the atrium 58 formed between the dual backplate skins 5a, 5b, the volume of which is larger than the volume of the inlet aperture 6b. It will be appreciated that constructing a laryngeal mask with a backplate 5 in which is formed an atrium or conduit 58 for gastric material is a highly efficient and economical way to use existing mask structures. Forming gastric drain tubes from an expandable material so that the space they occupy in the anatomy is minimised until they are called upon to perform their function is advantageous because it makes insertion of the device easier and causes less trauma to the delicate structures of the anatomy when the device is in place, particularly if the device is left in place for an extended period. And still further advantages are obtained if these features are combined such that the atrium 58 is formed from the soft material of the gastric drain tubes makes because the mask, whilst being sufficiently soft to avoid trauma on insertion can yet provide a large volume atrium 58 that can expand under pressure of vomiting. Such expansion results in a dorsal deformation of the outer skin 5b resembling a dome that acts like a spring against the back wall of the throat when the mask is in situ, forcing the cuff 6 against the larynx and thereby helping to maintain the device in its sealed state. The use of the device comprising connector 8 has the advantages that an air supply can be connected to the device from any desired position relative to the patient's face, the position of the air supply tube can moved once it is attached to allow access by the clinician, and the position of the device in the patient is not disturbed by movement of the air supply. The use of a device comprising fixation straps allows the device to be positioned very precisely by virtue of the hinges which provide multiple points of articulation and allow the position and degree of insertion to be tailored precisely to the patient's anatomy. In use, the device comprising an inner core 202 and an outer tube 201 provides strength to the airway tube. In addition, the combination of the inner core 202 and outer tube 201 provides one or more lumen which provides at least one conduit for the insertion of a viewing device or sensor, and/or for the passage of gastric matter.

Thus, it can be seen that the above described embodiments address the problems of prior art devices in novel and inventive ways.

Features of the above-described embodiments may be re-combined into further embodiments falling within the scope of the present invention. Further, the present invention is not limited to the exemplary materials and methods of construction outlined above in connection with the exemplary embodiments, and any suitable materials or methods of construction may be employed. For example, although the cuff may be formed using a sheet of soft flexible silicone rubber, other materials such as latex or PVC may be used. PVC as a material is particularly suited to embodiments intended for single use, whereas the use of silicone rubber is preferred although not essential for embodiments intended to be re-used in a number of medical procedures.

Further, and as would be appreciated by the skilled person, various features of the present invention are applicable to a wide range of different laryngeal mask airway devices, and the invention is not limited to the exemplary embodiments of types of mask described above. For example, aspects of the invention may be applied to laryngeal mask airway devices featuring epiglottic elevator bars over the mask aperture, which bars are operable to lift the epiglottis of a patient away from the aperture upon insertion of an endotracheal tube or other longitudinally-extended element inserted through the airway tube so as to emerge into the hollow or lumen of the mask through the mask aperture. Aspects of the present invention may for example be applied to single or re-useable devices, devices featuring aperture bars or not, “intubating” devices which permit an endotracheal tube or similar to be introduced into the larynx via an airway tube of a mask, devices incorporating fiberoptic viewing devices and so forth, without restriction or limitation on the scope of the present invention.

1. An artificial airway device to facilitate lung ventilation of a patient, comprising an airway tube including an airway lumen, a mask at one end of the airway tube, the mask including a backplate and having a peripheral formation capable of forming a seal around the circumference of the laryngeal inlet, the peripheral formation surrounding a hollow interior space or lumen of the mask and the airway lumen opening into the lumen of the mask, wherein the airway tube comprises an outer tube part and an inner core, the inner core defining partly or completely the airway lumen.

2. A device according to claim 1, wherein the inner core further defines partly or completely one or more additional lumen.

3. A device according to claim 2, wherein the one or more additional lumen is adapted to receive a sensor or viewing device.

4. A device according to claim 2 or 3, wherein the one or more additional lumen includes a recess for location of a sensor.

5. A device according to claim 2, 3 or 4, wherein the one or more additional lumen further includes one or more lumen disposed to allow in use, for access to the oesophageal sphincter of the patient and/or removal of gastric fluid.

6. A device according to any one of claims 2 to 5, wherein the or each additional lumen is defined entirely by the inner core, or by a combination of the inner core and the outer tube part.

7. A device according to any preceding claim, wherein at least one groove is provided on the outer surface of the inner core.

8. A device according to claim 7, wherein two grooves are provided on the outer surface of the inner core.

9. A device according to claim 7 or 8, wherein the at least one groove provided on the outer surface of the inner core and an inner wall of the outer tube form at least one lumen when the inner core is inserted into the outer tube part.

10. A device according to any preceding claim, wherein the airway tube has a fixed curve portion.

11. A device according to any preceding claim, wherein the outer tube part takes the form of a tube having a fixed curve portion.

12. A device according to any preceding claim, wherein the outer tube part comprises a backplate portion.

13. A device according to any preceding claim, wherein the outer tube part comprises a straight portion.

14. A device according to any preceding claim, wherein the outer tube part comprises a through bore running throughout from its proximal to distal ends.

15. A device according to claim 13 or 14, wherein the inner surface of the outer tube part comprises raised guide tracks.

16. A device according to claim 15, wherein the raised guide tracks extend from near the proximal end to the distal end of the straight portion.

17. A device according to any one of claims 13 to 16, wherein the outer surface of the inner core comprises at least one further groove.

18. A device according to claim 17, wherein the at least one further groove provided on the outer surface of the inner core fits with a corresponding track provided on the inner surface of the outer tube part.

19. A device according to any preceding claim, wherein the inner core is dimensioned to fit inside the outer tube part.

20. A device according to any preceding claim, wherein the inner core extends substantially along the length of the outer tube part.

21. A device according to any preceding claim, wherein the inner core comprises an inner backplate portion.

22. A device according to any preceding claim, wherein the airway tube comprises silicone or PVC.

23. A device according to any preceding claim, wherein the outer tube part and/or the inner core comprise silicone or PVC.

24. A device according to any one of claims 3 to 23, wherein the sensor is a temperature sensor.

25. A device according to claim 24, wherein the temperature sensor is provided on the airway tube.

26. A device according to claim 25, wherein the temperature sensor is provided on the inner core.

27. A device according to claim 25, wherein the temperature sensor is provided on the outer tube part.

28. A device according to any one of the preceding claims, wherein the peripheral formation comprises an inflatable cuff, or a non-inflatable cuff.

29. A device according to claim 28, wherein when the peripheral formation comprises an inflatable cuff, the backplate overlies the cuff and is bonded to it, such that on deflation the cuff may be collapsed upon it, thereby encouraging the cuff to pack flat.

30. A method of treating a patient using a device according to any one of claims 1 to 29.

31. A method of manufacturing an airway tube for a laryngeal mask airway device, the method comprising the steps of forming an airway tube by providing an inner core and an outer tube part, the inner core defining an airway lumen, and inserting the inner core into the outer tube part.

32. A method according to claim 31, including the step of forming the inner core by injection moulding.

33. A method according to claim 31, including the step of forming the inner core by extrusion.

34. A method according to claim 31, 32 or 33, including the step of forming the outer tube by injection moulding.

35. A method according to claim 31, 32 or 33, including the step of forming the outer tube by extrusion.
